(19) United States
(12) Patent Application Publication
(10) Pub. No.: US 2006/0004394 A1
(43) Pub. Date: Jan. 5, 2006

(54) Double-ended conduit with graded locking sleeves

(76) Inventor: Paul D. Amarant, Davie, FL (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/121,611

(22) Filed: May 4, 2005

Related U.S. Application Data
(60) Provisional application No. 60/570,660, filed on May 13, 2004.

(51) Int. Cl. A61B 17/08

(52) U.S. Cl. 606/153

(57) ABSTRACT

An apparatus for joining two vessels in end-to-end anastomosis comprises a hollow tubular conduit having two ends, and a pair of opposing locking sleeves disposed along an outer surface of the conduit and at least partially moveable thereover. At least one of the conduit ends may include a grasping member, such as a barbed ring, adjacent the end. Each of the locking members has an inner diameter related to the outer diameter of a respective conduit end such that a vessel positionable between said locking member and said conduit end is substantially locked thereby, to inhibit disengagement of said vessel end from said apparatus.

### RELATED APPLICATION

The present patent document claims the benefit of the filing date under 35 U.S.C. §119(e) of Provisional U.S. Patent Application Ser. No. 60/570,660, filed May 13, 2004, which is hereby incorporated by reference.

### BACKGROUND

1. Technical Field

The present invention relates to a tubular member for use in joining attachable tissue segments, and more particularly, to a tubular conduit for end-to-end anastomosis of medical grafts, body vessels, and the like.

2. Background Information

Anastomosis is the joinder of hollow vessels to create an internal communication between them. An anastomosis is generally created by a surgical procedure that joins two body vessels, vascular grafts, or a body vessel and a graft, in order to create or restore a pathway for fluid flow through the joined structure. Commonly, an anastomosis is created by vascular surgery to join two blood vessels, grafts, or a blood vessel and a graft, to create or restore blood flow therethrough.

Current devices and techniques exist which allow for open-ended surgical attachment of harvested blood vessels or grafts for purposes such as the avoidance of a vessel blockage, replacement of diseased vessels, and vascular access. Such techniques include sewing or otherwise attaching a vessel or graft between open ends of existing vessels. Examples of vessel pairs which are frequently joined by a vessel or graft include an internal mammary artery and a coronary vessel, the radial artery and cephalic vein, the brachial artery and cephalic vein, the brachial artery and basilica vein, the ulnar artery and a basilica vein, and a brachial artery and branches of the antecubital vein, among others.

It is generally preferred to join such vessels utilizing the patient's natural vessels. This connection may be between two natural vessels positioned in their natural place of orientation in the body, or alternatively, utilizing one or more natural vessels harvested from another portion of the patient's anatomy. Utilizing a vessel harvested from another portion of the patient's anatomy minimizes the possibility that the patient will experience incompatibility or rejection problems of the type that may occur when using graft materials that originate from an external source, or from using exogenous tissue. In addition, such harvested vessels provide a ready supply of biological tissue that has already proven to be biologically compatible with the patient.

At times, however, suitable body vessels may not be available for harvesting. In such cases, a synthetic vessel (e.g., TEFLON® or DACRON®) or an exogenous vessel may be used. Synthetic vessels have been found to be effective in many instances. However, such vessels have shown a greater propensity to become narrowed than do natural arteries or veins. Exogenous vessels may also be utilized in an appropriate case. However, there is a greater likelihood of patient incompatibility with such vessels when compared to vessels harvested from the patient.

Many different types of anastomosis connections between a vessel and a graft are known in the medical arts. For example, an anastomosis connection may be utilized to join vessels from the end of a graft to the side of a vessel, commonly referred to as an end-to-side connection. An anastomosis connection may also be utilized to join the end of a graft to the end of a vessel, commonly referred to as an end-to-end connection. A side-to-side connection of a vessel and a graft may also be established. This type of connection is commonly referred to as a fistula.

End-to-end connections are generally considered beneficial because they essentially mimic the normal flow of fluid through the natural vessel. With regard to such end-to-end connections, however, it is important to insure that a secure and leak-free connection be established. Prior art connection devices are at times unsecure, and also have been prone to leakage. While synthetic joinder materials are available, such materials are often complicated and difficult to use. A need exists for an improved device for end-to-end connection that provides a secure and leak-free connection, that is relatively easy for the surgeon to manipulate and insert, and that is cost-effective.

### BRIEF SUMMARY

The present invention addresses the problems of the prior art by providing an improved conduit for an end-to-end anastomosis connection. The conduit may comprise a double-ended conduit having graded locking sleeves.

In one embodiment, the present invention comprises an apparatus for use in end-to-end anastomosis. The apparatus comprises a hollow tubular conduit having two ends, and a pair of opposing locking sleeves disposed along an outer surface of the conduit. At least one of the ends may include a barbed ring adjacent the end. Each of the locking sleeves has an inner diameter related to an outer diameter of the conduit in a manner such that little or no clearance extends therebetween.

In another embodiment, the present invention comprises a method for end-to-end anastomosis of vessels. An anastomosis device includes a hollow tubular conduit having two ends, a barbed ring adjacent at least one of the ends, and first and second locking sleeves disposed along an outer surface of the conduit. Each of the locking sleeves has an inner diameter related to an outer diameter of the conduit such that little or no clearance extends therebetween. A first vessel is slid in axial direction over one of the ends of the conduit and over an adjacent barbed ring toward a center portion of the conduit. A second vessel is then slid axially from the opposite side of the conduit over the other end of the conduit toward a center portion of the conduit. The first vessel is further slid axially toward the center portion between the first locking sleeve and the conduit outer surface, such that the first vessel frictionally engages the first locking sleeve and the conduit outer surface. The second vessel is further slid axially toward the center portion between the second locking sleeve and the conduit outer surface, such that the second vessel frictionally engages the second locking sleeve and the conduit outer surface. The first and second sleeves may then be locked around the respective first and second vessels, such as by sliding the sleeves axially in a direction away from the center portion of the conduit.

### BRIEF DESCRIPTION OF THE DRAWINGS

FIG. 1 is a side view, partially in section, of an embodiment of the double-ended conduit of the present invention;

FIG. 2 is a sectional view of a locking sleeve of the apparatus of FIG. 1;

FIG. 3 is an end view of the locking sleeve of FIG. 2;

FIG. 4 is a side view, partially in section, of the double-ended conduit of FIG. 1, showing the joinder of two vessels;

FIG. 5 is a view of the conduit of FIG. 4, showing the locking sleeves in a locked position;

FIG. 6 is a side view, partially in section, of an alternative embodiment of a double-ended conduit;

FIG. 7 is a view of the conduit of FIG. 6, showing the locking sleeves in a locked position; and

FIG. 8 a side view, partially in section, of another alternative embodiment of the invention.

### DETAILED DESCRIPTION OF THE DRAWINGS AND THE PRESENTLY PREFERRED EMBODIMENTS

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The present invention comprises a percutaneous anastomosis connection system for establishing an end-to-end anastomosis connection between two hollow structures in the body. The particular connections resulting from use of the inventive apparatus may be, for example, a graft-to-graft connection, a vessel-to-graft connection, or a vessel-to-vessel connection. For a vessel-to-vessel connection, the connection may be established between natural vessels, exogenous vessels, synthetic vessels, or any combination of the foregoing.

Although it is expected that the apparatus will normally be utilized to connect blood vessels, other body vessels may be joined to vessels, other body structures, grafts, synthetic or exogenous vessels. One non-limiting example of a connection of this type comprises the connection of the ureter vessel to the urethra. For applications in bodily systems, such as the circulatory system, ultrasound guidance can be utilized to help establish connection between the apparatus and other structures in the system, such as an artery, a vein, or both an artery and a vein. Those skilled in the art will appreciate that other bodily connections can be made using the apparatus and method of the present invention, and that medical guidance systems other than ultrasound may be utilized in an appropriate case, all of which are considered within the scope of the invention.

Such hollow body vessels, such as blood vessels, are joined in a manner to permit or restore fluid flow therebetween. The anastomosis connection provides a means to bridge the vessels within the body of a patient in end-to-end fashion. The term “vessel” is used herein in inclusive fashion to include body vessels or other hollow structures (both endogenous and exogenous), medical grafts, synthetics, and other segments that may be joined by the apparatus of the present invention.

In a preferred embodiment, the inventive apparatus for establishing the end-to-end anastomosis connection comprises a double-ended tubular conduit. The tubular conduit is provided with a plurality of locking sleeves for locking the vessels, etc., to be joined. Preferably, the locking sleeves are oriented such that a separate locking sleeve is provided at each axial end of the conduit. The double-ended tubular conduit may also include one or more grasping elements, such as a barbed ring, disposed near each axial end of the tube. Preferably, the locking sleeves have a graded internal diameter that decreases in the direction of the center of the tubular conduit.

The invention will now be described in connection with the figures. FIG. 1 illustrates one embodiment of an apparatus 10 for establishing an end-to-end anastomosis of medical grafts, blood vessels or other hollow body structures. Apparatus 10 comprises a generally cylindrical hollow tubular conduit 12 having opposing axial ends 14, 16. Tubular conduit 12 preferably includes one or more grasping structures, such as barbed rings 18, 20 positioned along the circumference of conduit 12. Each one of opposing ends 14, 16 is disposed on a separate side of an imaginary midline 25 that separates tubular conduit 12 into two half-sections, or ends.

Apparatus 10 also includes opposing sleeves 24, 26 circumferentially disposed on the outer surface of tubular conduit 12. Each one of sleeves 24, 26 is preferably disposed on a separate side of imaginary midline 25. Preferably sleeves 24, 26 have a graded internal diameter. Sleeve 24 is aligned such that the internal diameter of the sleeve decreases toward the center of the conduit (i.e., toward imaginary center line 25), from a maximum diameter end 28 to a minimum diameter end 30. Sleeve 26 is aligned such that the internal diameter of the sleeve decreases toward the center of the conduit, from a maximum diameter end 29 to a minimum diameter end 31.

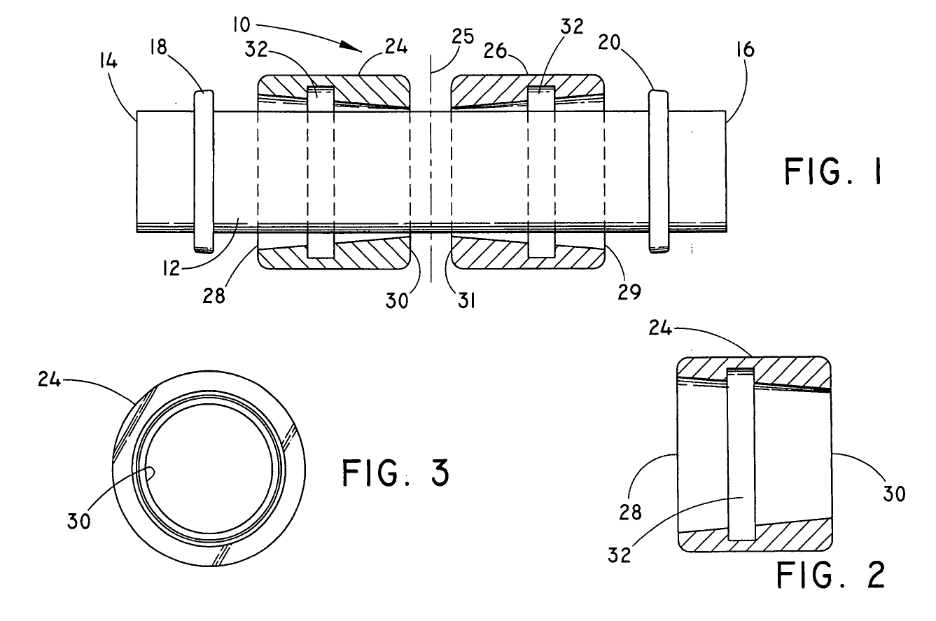

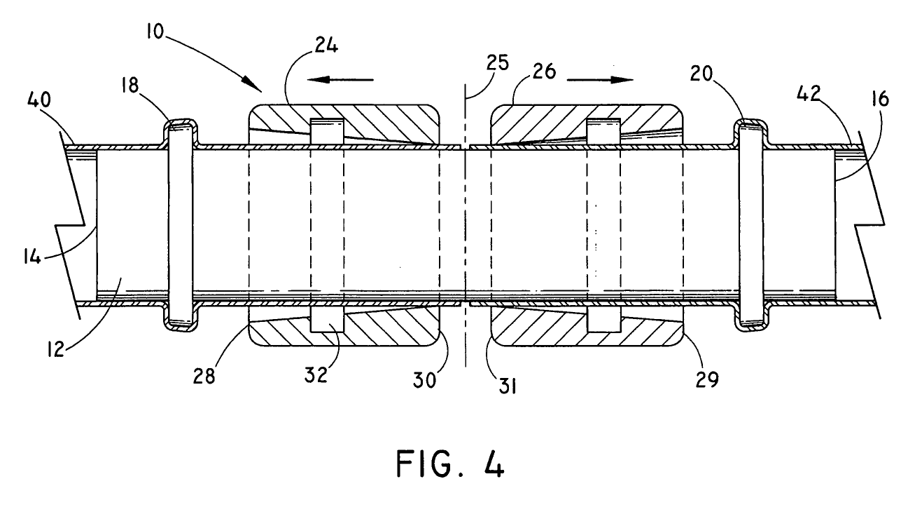

FIG. 2 is a cross-sectional view of sleeve 24 showing diameters 28, 30. FIG. 3 is an end view of sleeve 24, taken from the orientation of FIG. 1. The outer diameter of conduit 12 is sized relative to the minimum inner diameter 30, 31 of sleeves 24, 26 such that there is little or no clearance between the conduit outer diameter and the sleeve minimum diameter.

Use of apparatus 10 for establishing an anastomosis connection between two vessels will now be described. Initially, tubular conduit 12 is loaded by inserting graded sleeves 24, 26 onto respective conduit ends 14, 16. Since there is little or no clearance between the conduit outer diameter and the sleeve minimum diameter, the tubular conduit must normally be flexed, squeezed or otherwise momentarily deformed to allow the locking sleeves to pass over respective grasping structure 18, 20, such as barbed rings, to reach the positions shown in FIG. 1. The compositions of tubular conduit 12, grasping structures 18, 20 and sleeves 24, 26 are selected to enable the apparatus 10 to be assembled in this manner. Alternatively, the various components of apparatus 10 may be loaded by other means well known in the art.

As shown in FIG. 4, a first body vessel 40 to be joined is slid in an axial direction (to the right in FIG. 4), over end 14 and barbed ring 18 of tubular conduit 12, in the direction of the imaginary midline 25. A second vessel 42 is then slid in an opposite axial direction (to the left in FIG. 4), over end 16 and barbed ring 20 in the direction of imaginary midline 25. Each one of locking sleeves 24, 26 is then urged axially in the direction of the arrows over the ends of respective vessels 40, 42 as far as they can slide, to tighten, or lock, the vessel in place. The lack of significant clearance between the conduit outer diameter and the sleeve minimum diameter provides a frictional force that locks, or otherwise pins the vessel end in place between the tubular conduit and each of the respective sleeves, thereby preventing the vessel from migrating.

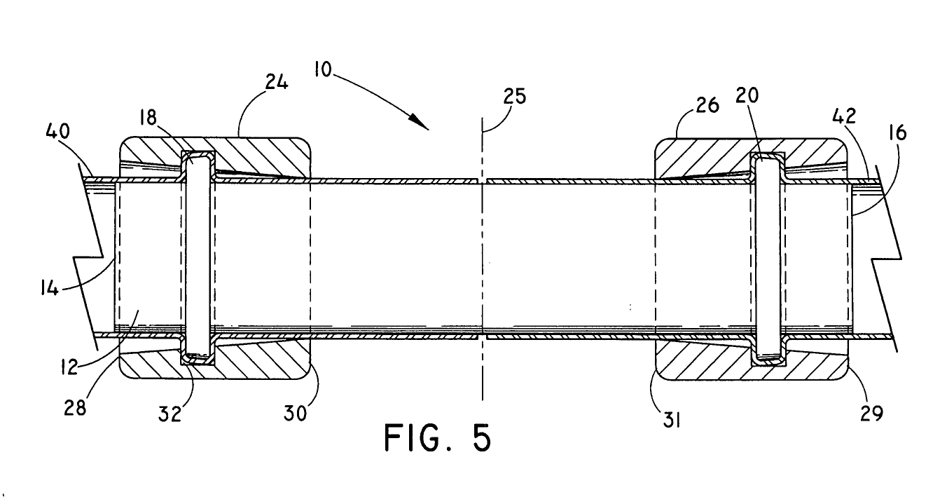

If desired, either or both of sleeves 24, 26 can be provided with an additional locking feature to better secure the locking of the vessel to the conduit. In one embodiment, the locking feature comprises a cut-out portion, such as notch 32, that extends circumferentially along all or part of the circumference of the inner surface of the sleeve. Notch 32 is best shown in FIG. 2. Notch 32 can be used to assist in tightly holding, or locking, the sleeves on the barbs or other grasping structures. In the embodiment of FIG. 4, respective sleeves 24, 26 can be slid axially in the direction of the arrows until they reach respective barbed rings 18, 20. Preferably, the sleeve can be locked and securely held in position by the interconnection of notch 32 with the respective ring 18, 20, as shown in FIG. 5.

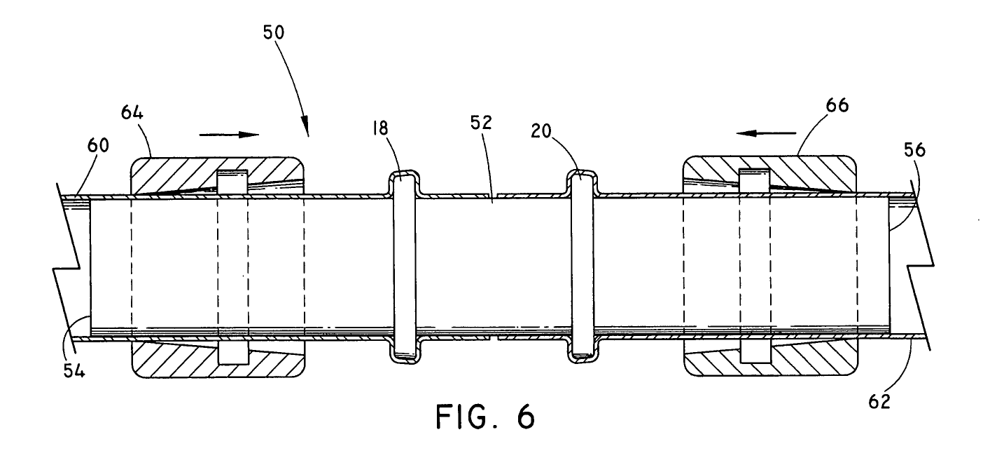

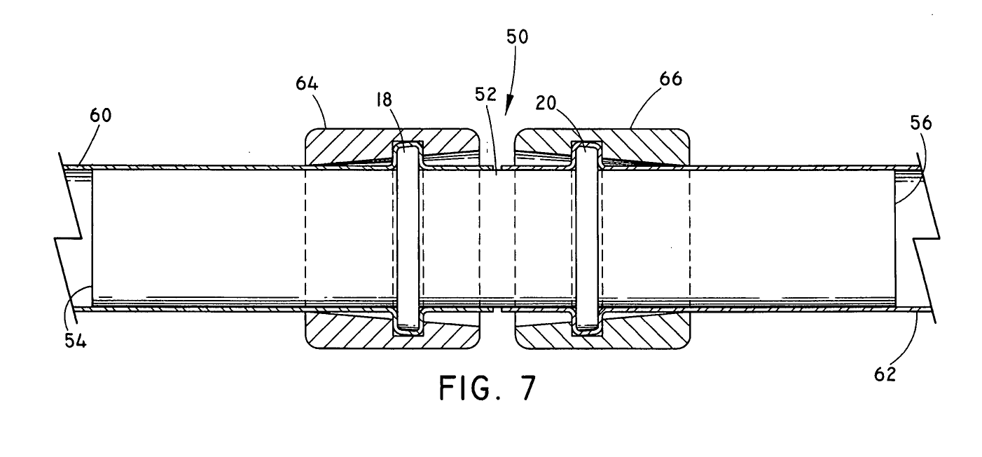

An alternative embodiment of an apparatus 50 for establishing an end-to-end connection is shown in FIG. 6. This embodiment includes tubular conduit 52 having axial ends 54, 56. A respective graded sleeve 64, 66 is positioned at each axial end 54, 56 of the conduit. In this embodiment, the sleeves may be placed around the axial ends 60, 62 of the conduit to fasten the vessel or graft in place. Alternatively, a projection, such as a flange, may be incorporated onto each axial end of conduit 52 to secure the connection. A notch can be incorporated into either or both of the graded sleeves as disclosed in the previous embodiment, to receive the projection, or to receive grasping structures 18, 20, as shown in FIG. 7. Those skilled in the art will appreciate that other conventional attachment mechanisms may be substituted for those shown, the objective being to securely lock the vessel portion between the sleeve and the conduit.

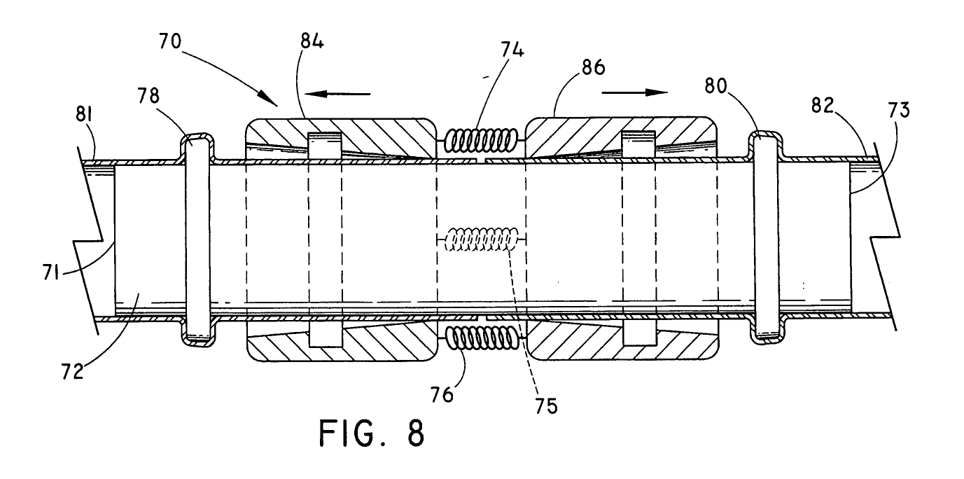

Yet another alternative embodiment of a connection apparatus 70 is shown in FIG. 8. Apparatus 70 includes tubular conduit 72, axial ends 71, 73, barbed rings 78, 80, and sleeves 84, 86. In this embodiment, one or more springs 74, 75, 76 are provided between sleeves 84, 86 to interconnect the sleeves. The embodiment shown in FIG. 8 includes four springs (one of which is not visible in the sectional view of FIG. 8) spaced about 90 degrees from each other along the circumference of tubular conduit 72. Those skilled in the art will appreciate that more, or fewer, springs may be used in an appropriate case. The springs may be compressed during loading of the vessels 81, 82 on tubular conduit 72, but preferably have a tendency to elongate in the axial direction to lock the vessel ends on conduit 72. If desired, sleeves 84, 86 can be further secured on barbed rings 78, 80, as illustrated in previous embodiments. In an alternative embodiment, springs can be provided having a tendency to compress in the axial direction.

All components described herein are formed of biologically compatible conventional materials having sufficient strength for the purposes described. Preferably, the tubular conduit is formed of a rigid or semi-rigid plastic, of a type suitable for implantation into a human or other animal.

Although the inventive apparatus may be conveniently used to join two blood vessels, those skilled in the art will recognize that other known components can likewise be joined, such as synthetic graft material and exogenous materials. Likewise, a blood vessel may be attached to a synthetic graft vessel or an exogenous vessel. Furthermore, the invention is not limited to vascular access, but rather, may also include the applications such as bypass grafting between two blood vessels, including fem-fem (femoral artery and femoral vein) and fem-pop; coronary artery bypass grafting; and shunting outside of the circulatory system to help alter flow of fluid including gastrointestinal tract (e.g., liver and gall bladder), the urinary system (e.g., ureter and urethra), beyond the blood-brain barrier (e.g., for hydroencephalopathy), and in the reproductive system (e.g., ovarian recannulation).

It is therefore intended that the foregoing detailed description be regarded as illustrative rather than limiting, and that it be understood that it is the following claims, including all equivalents, that are intended to define the spirit and scope of this invention.

1. An apparatus for joining two vessels in end-to-end anastomosis, comprising:
a tubular conduit, said tubular conduit having two ends, each end having an outer diameter, the outer diameter of each end sized such that one of said vessels to be joined is positionable over a separate one of said two ends; and
a pair of opposing locking members disposed along an outer surface of said tubular conduit and at least partially movable thereover, each of said locking members having an inner diameter, wherein said respective inner diameter of each of said locking members is related to said outer diameter of said conduit end such that disengagement of a vessel positionable between said locking member and said conduit is inhibited.

2. The apparatus of claim 1, wherein one of said locking members is disposed at each of said conduit ends, further comprising a grasping element disposed on at least one of said conduit ends, said grasping element adapted for grasping said vessel positioned over said at least one end.

3. The apparatus of claim 2, wherein said grasping element comprises a barb.

4. The apparatus of claim 2, wherein a grasping element is disposed at each of said ends.

5. The apparatus of claim 4, wherein at least one of said grasping elements comprises a barbed ring disposed substantially around a circumference of said conduit.

6. The apparatus of claim 1, wherein each of said locking members is disposed on a separate side of an imaginary line at an axial midpoint of said tubular conduit.

7. The apparatus of claim 2, wherein at least one of said locking members comprises a locking sleeve, and wherein said sleeve has a graded internal diameter.

8. The apparatus of claim 7, wherein said graded internal diameter of said at least one locking sleeve decreases from a large diameter portion to a small diameter portion in the direction of an axial midpoint of the tubular conduit, said small diameter portion sized relative to said outer diameter of said conduit end to inhibit said disengagement of said vessel.

9. The apparatus of claim 8, wherein said at least one locking sleeve further comprises a notch disposed along said graded internal diameter, said notch sized such that at least a portion of said grasping element is receivable therein.

10. The apparatus of claim 8, wherein said at least one locking sleeve comprises two locking sleeves, each of said locking sleeves comprising a notch disposed along said graded internal diameter and sized such that at least a portion of a grasping element is receivable therein.

11. The apparatus of claim 7, wherein each of said locking members comprises a locking sleeve.

12. The apparatus of claim 11, further comprising a grasping element disposed at each of said ends, wherein each of said grasping elements comprises a barbed ring, each of said sleeves comprising a notch sized such that at least a portion of said barbed ring is receivable therein.

13. The apparatus of claim 1, further comprising at least one spring member disposed in an axial space between said opposing locking members and in engagement with said locking members.

14. The apparatus of claim 7, wherein each of said locking members comprises a locking sleeve, further comprising at least one spring engaging each of said locking sleeves and exerting spring tension therebetween, said at least one spring having a tendency to elongate away from an axial midpoint of said tubular conduit.

15. The apparatus of claim 14, further comprising a plurality of springs, each of said springs disposed in a space between said locking sleeves and in engagement therewith, each of said springs having a tendency to elongate away from said axial midpoint.

16. A method for joining two vessels in end-to-end anastomosis, comprising:
providing an apparatus comprising a hollow tubular conduit having two ends and an outer surface, and having first and second locking sleeves disposed along said outer surface, each of said locking sleeves having an inner diameter related to an outer diameter of said conduit to inhibit disengagement of a vessel end positionable therebetween;
sliding a first one of said vessel ends axially over one of said ends of said conduit toward a center portion of said conduit, and sliding said first locking sleeve over said first vessel end to position said first vessel end between said first locking sleeve and said conduit outer surface; and
sliding a second one of said vessel ends axially over the other of said ends of said conduit toward a center portion of said conduit, and sliding said second locking sleeve over said second vessel end to position said second vessel end between said second locking sleeve and said conduit outer surface.

17. The method of claim 16, wherein said hollow tubular conduit includes a grasping element on at least one of said conduit ends, said grasping element sized and configured for grasping said vessel.

18. The method of claim 17, wherein said hollow tubular conduit includes a grasping element at each of said conduit ends.

19. The method of claim 18, wherein at least one of said grasping elements comprises a barbed ring.

20. The method of claim 18, wherein each of said locking sleeves includes a notch sized such that at least a portion of one of said grasping elements is receivable in said notch, said method further comprising sliding said first locking sleeve such that at least a portion of one of said grasping elements is received therein, and sliding said second locking sleeve such that at least a portion of the other of said grasping elements is received therein.
